(19) United States
(12) Patent Application Publication
(10) Pub. No.: US 2023/0225852 A1
(43) Pub. Date: Jul. 20, 2023

(54) AORTIC ROOT REPLACEMENT GRAFT AND SURGICAL METHOD

(71) Applicant: Sutherland Cardiovascular Ltd., London (GB)

(72) Inventor: Fraser William Havern SUTHERLAND, London (GB)

(73) Assignee: Sutherland Cardiovascular Ltd., London (GB)

(21) Appl. No.: 17/663,369

(22) Filed: May 13, 2022

Related U.S. Application Data
(63) Continuation of application No. 29/823,577, filed on Jan. 18, 2022.

(51) Int. Cl. A61F 2/06; A61F 2/95

(52) U.S. Cl. CPC A61F 2/064; A61F 2/95; A61F 2250/0097

(57) ABSTRACT

Aortic root replacement grafts, systems and methods for implanting an aortic root replacement graft are disclosed. The graft includes a tubular portion; a bulged portion with a first end and a second end, wherein the second end of the bulged portion is coupled to a first end of the tubular portion; a cuff portion coupled to the first end of the bulged portion; and at least one side arm coupled to and extending from the bulged portion. Methods for implanting an aortic root replacement graft with two side arms are also disclosed.

### CROSS REFERENCE TO RELATED APPLICATIONS

This application is a continuation of U.S. Design patent application Ser. No. 29/823,577 filed on Jan. 18, 2022 and entitled Aortic Root Replacement Graft, which is incorporated herein by reference in its entirety.

### TECHNICAL FIELD

The present disclosure relates generally to general, heart, and artery surgery related to aortic root defects. More specifically, but not exclusively, the present disclosure relates to aortic root grafts and methods for performing aortic root replacement surgery.

### BACKGROUND OF THE INVENTION

Aortic root replacement (ARR) is an operation performed by heart surgeons to replace the aortic root, i.e. that portion of the ascending aorta that is exits the heart, incorporates origins of the right and left coronary arteries and supports the leaflets of the aortic valve.

### Aortic Root Replacement in USA

Sotiris Stamou and associates have previously sought to evaluate early clinical outcomes of aortic root surgery in the United States of America and published their findings in the Journal of Thoracic and Cardiovascular Surgery in January 2015. In this study, the authors interrogated the Society of Thoracic Surgeons database to identify all patients who had undergone aortic root replacement from 2004 to early 2010. A total of 13,743 cases were thus reported. The median age was 58 years (range, 18-96 years); 3,961 were women (29%) and 12,059 were white (88%). The different aortic root surgery procedures included placement of a mechanical valve conduit (4,718 cases, 34%), stented pericardial (4,879 cases 6.4%) or porcine bioprosthesis (478 cases, 3.5%), stentless root (4,309 cases, 31%), homograft (498, 3.6%), and valve sparing root replacement (1,918 cases, 14%).

An interesting and important finding was that most cardiac centers performed aortic root surgery in very small volumes. The median number of aortic root surgeries per site was two, and only 5% of sites performed more than 16 aortic root surgeries annually. This is pertinent because aortic root replacement surgery in its current form is a technically demanding procedure.

Aortic root replacement is firstly indicated when the aortic root is severely dilated and presents a risk of aortic dissection and/or frank aortic rupture with consequent risk to life.

Aortic root replacement is secondly indicated when dilatation of the aorta interferes with aortic valve function, typically rendering the valve incompetent and causing symptoms and/or signs of heart failure with an attendant, symptoms and reduced life expectancy.

During aortic root replacement surgery, the native aortic root is typically excised in its entirety and replaced with a substitute aortic root, most commonly made from an inert, artificial graft material.

Artificial aortic root replacement grafts are typically provided in a range of radii or diameters for selection by the surgeon.

The diameter of the graft selected for an individual patient by the surgeon is most commonly determined by the measured diameter of the native aortic annulus and/or the diameter of the aortic valve or the diameter of the selected aortic valve prosthesis when decision has been made to replace the aortic valve. Valve sizers and graft sizers are used for this purpose.

It is pertinent that the diameter of the native enlarged aortic root is rarely a consideration in selection of graft size. Sizing of the aortic annulus or various measures of the aortic valve itself, like leaflet height for example, take clear precedence over sizing of the native diseased aorta itself in the selection of an appropriately sized aortic root replacement graft.

As with all surgery the recommendation for aortic root replacement is based upon a balanced judgement of benefit versus risk. There are published guidelines to help guide surgeons and their patients in decision making, as regards aortic root replacement surgery. Such guidelines typically provide generally accepted criteria for operative intervention based upon aortic root dimensions, aortic valve functional morphology and clinical and investigational markers of heart function, both short and long term.

Examples are (1) the 2014 ESC Guidelines on the diagnosis and management of aortic diseases and (2) the 2010 ACCF/AHA/AATS/ACR/ASA/SCA/SCAI/SIR/STS/SVM Guidelines for the Diagnosis and Management of Patients with Thoracic Aortic Disease.

Aortic root replacement is a technically demanding surgical procedure with attendant risk of peri-operative mortality and morbidity.

Aside from death, known risks of heart surgery in general include stroke, localised or systemic infection and major organ dysfunction.

Risks pertaining especially to aortic root replacement surgery include (1) acute bleeding at the time of surgery, either from suture lines, cannulation sites or from surrounding tissues, (2) late pseudoaneurysm formation from one or more of the suture lines created during the aortic root replacement operation and (3) coronary ischaemia caused by kinking, tension and/or distortion of the coronary artery buttons where they are anastomosed to the aortic root replacement graft.

It is pertinent that bleeding and coronary ischemia do not merely pose an inconvenience to the surgeon but are potentially life-threatening complications for the patient so these complications must be taken very seriously. It follows that, improvements in graft technology or methods of implanting the same, however minor, will naturally be welcomed by surgeons and patients alike and therefore have usefulness.

It is an intended purpose of the current disclosure to overcome problems of current aortic root replacement grafts and current means of implanting aortic root replacement grafts by (1) making the surgery technically easier to perform for example by obviating the need for coronary artery mobilisation, (2) ensuring that the suture lines are more accessible at the end of the procedure so rendering haemostasis easier to confirm and also easier to confirm that abutment of graft to native tissues is secure making late pseudoaneurysm formation less likely, (3) rendering kinking, tension and distortion of the coronary arteries potentially created by the coronary button to graft anastomosis less likely, (4) make distal anastomosis more secure in terms of acute control of bleeding and reduction in the risk of late false aneurysm formation that can occur at an unsupported distal anastomosis, (5) providing an improved means of venting air from the heart at the end of the procedure that may be more quickly and more easily closed with little risk of bleeding therefrom, and (6) provide a sizer for better orientation of aortic root replacement graft substitute that is equipped with side arms for anastomosis with coronary buttons.

### More Detail on Aortic Root Replacement

Aortic root replacement operations may include replacement of the entire ascending aorta up to or even beyond junction with the aortic arch so aortic root replacement grafts typically have to be trimmed to an appropriate length by the operating surgeon.

Aortic root surgery operations may require excision and replacement of the native aortic valve with a prosthetic valve.

Alternatively, aortic root surgery operations may be performed with preservation of the native aortic valve, so called valve sparing aortic root replacement.

When the surgeon chooses to replace the native aortic valve, the selected valve prosthesis may be a mechanical type of valve or a biological type of valve. These may be provided as separate standalone devices for the surgeon to sew into an aortic root replacement graft or else may be provided as a composite wherein the manufacturer supplies the surgeon with a prosthetic valve with aortic root replacement graft already attached. The latter obviates the need for the surgeon to attach the prosthetic valve to the aortic root replacement graft with obvious time advantage. Attachment of the graft to the prosthetic valve during manufacture also provides a more reliable means of attachment reducing the risk of acute bleeding and/or late pseudoaneurysm formation at the particular suture line located between the graft and the prosthetic valve is reduced.

Typically, grafts are attached inside the sewing cuff of the valve prosthesis which makes for an aortic root replacement graft diameter somewhat smaller than the maximal diameter of the aortic valve prosthesis. This is of more than theoretical concern because, as explained below, there is already a natural size discrepancy between the size of said attachment of graft to valve during manufacture whilst conferring the obvious advantages explained above, has the effect of accentuating the size discrepancy between the maximum diameter of the selected graft and the diameter of the native diseased aorta.

### Attachment of Coronary Arteries to Graft

The current method of aortic root replacement operation requires reattachment of the coronary arteries to a straight graft or to the bulged portion of an aortic root replacement graft of the types: Valsalva type (Terumo Aortic) or Cardioroot type (Getinge).

The Valsalva Graft from Terumo Aortic and Cardioroot Graft from Getinge incorporate a bulged profile towards one end of the graft to increase the diameter of the graft to be closer to the diameter of the diseased native aorta at the level of the coronary arteries. However, significant size discrepancy still exists leaving gaps between the coronary arteries and the bulged portion of the graft. These gaps must be closed by mobilisation of the coronary arteries in order that the coronary arteries meet the graft.

It should be noted that the currently available bulge type grafts also benefit from the known benefits on aortic valve function of replication of the sinuses of Valsalva displayed in the Valsalva Graft from Terumo Aortic and Cardioroot from Getinge.

In order for the coronary buttons to meet the graft, the, mobilisation of the coronary arteries, selection of site for attachment of coronary buttons to the graft and fenestration of the graft with an electric Bovie or equivalent, thus allowing anastomosis of each coronary artery to the graft at the two fenestrations.

### Role of Judgement and Experience in Aortic Root Replacement

All of the above steps must be carefully performed. Safe execution of these steps requires expertise and judgment. Judgment and expertise are improved by experience. However, as explained in the opening paragraphs, case numbers in many centres are known to be low. Therefore, there is a pressing need for alternative means that make these steps easier to perform and thereby diminish the importance of expertise and judgment in their safe execution.

### Aortic Dimensions in the Diseased State and Relevance to the Location of Coronary Arteries

By way of background the normal diameter of the ascending aorta is 29±3 mm in males and 26±3 mm in females. At the sinus level, normal aortic dimensions are 34±3 mm in males and 30±3 mm in females (Table 1 from Erbel et al. 2001).

As explained in the opening paragraphs the commonest indication for surgery is dilatation of the aortic root and/or ascending aorta. In terms of actual dimensions, an ascending aortic diameter of 55 mm is typically considered the threshold for surgical intervention irrespective of aetiology and the presence or other wise of concomitant aortic valve disease or dysfunction [Ref. 2014 ESC Guidelines on the diagnosis and treatment of aortic diseases, page 2903 and 2010 AHA Guidelines for the Diagnosis and Management of Patients With Thoracic Aortic Disease, page e320].

Patients with an aortic diameter that falls short of the commonly accepted 55 mm threshold for intervention may still merit aortic root replacement if the aortic valve is bicuspid, or the valve exhibits significant pathology either aortic valve stenosis or aortic valve incompetence.

It is pertinent that the 55 mm measurement is a threshold for intervention, thus it will be appreciated that patients coming to surgery will generally have aortic dimensions greater than this, typically in the peri 60 mm range and sometimes much more.

Now turning attention to the maximum dimensions of aortic valve prostheses currently available, examples of the largest size of mechanical aortic valve prostheses currently on the market, from leading manufacturers in the field, are 27/29 mm (On-X, Artivion), 31 mm (Open Pivot Mechanical Aortic Heart Valve, Medtronic), 31 mm (Carbomedics Standard, Corcym).

When the surgeon chooses to attach the graft to the aortic valve prosthesis during aortic root replacement surgery, a graft is typically selected whose diameter is just few mm greater than that of the sewing cuff on the valve in order to ensure that the anastomosis created between the valve and the graft does not leak blood acutely and is not subject to late false aneurysm formation. As explained in the opening paragraphs, the dimensions of the aortic valve or aortic valve prosthesis take precedence over the diameter of the native diseased aorta during sizing.

Considering some practical examples, if the surgeon selects a 27/29 mm valve prosthesis, the largest diameter aortic valve prosthesis on the market On-X from Artivion, and then goes on to select an aortic root replacement graft that is a few mm larger then, at most the graft will measure 32 mm in diameter, thus it will be observed that there remains a considerable size discrepancy between the selected 32 mm graft and the threshold 55 mm diseased native aorta of a patient.

Moreover, when the surgeon chooses to avoid anastomosis of the valve prosthesis to the graft by choosing to use a pre-assembled valved graft on account of the known advantages of pre-assembled valved conduits, it will be observed, by way of example, that the diameter of the main bore of the graft on the pre-assembled valved conduit attached to the 27/29 mm On-X valve on the 27/29 mm Ascending Aortic Prosthesis from Artivion is just 26 mm, even smaller than if the surgeon were to assemble the graft and valve in the operating room. Once again, drawing attention to the discrepancy in size between the main bore of the graft size and the 55 mm threshold aortic diameter for surgery.

Thus, whether the surgeon chooses to attach the prosthetic valve to the graft during the procedure or selects a pre-assembled valved graft, there is a significant size discrepancy between the diameter of the selected graft and the diameter of the native ascending aorta and aortic root.

The dimensional constraints outlined above for mechanical aortic valve prostheses and associated conduits are the same for biological aortic valve prostheses and biological valved conduits.

Hence the diameter of grafts attached to the valved conduits falls far short of the diameter of the diseased ascending aorta and aortic root.

There is in turn a size discrepancy between graft and position of the origins of the coronary arteries in the aortic root replacement operation.

Turning now to the aortic root replacement operation, the surgeon is required to attach the coronary arteries with a cuff or surrounding aortic wall, the so called ‘coronary buttons’ to the sides of the aortic root replacement graft substitute.

In order to overcome this dimensional discrepancy outlined above, the coronary buttons must either be drawn towards the graft and anastomosed under tension, an approach that has historically been associated with bleeding and/or coronary ischaemia and very poor operative outcomes or else the arteries must be dissected from surrounding tissues, that is ‘mobilised’, in order that they can be brought safely into proximity of the graft for tension free anastomosis. Mobilisation of the coronary buttons is the standard, prior art technique practised today.

Just to reiterate the problem of not mobilising the coronary buttons or of inadequate mobilisation of the same, tension in a vascular anastomosis is known predispose to acute bleeding and/or late aneurysm formation at the anastomotic suture line between the coronary button and the graft. Tension may also precipitate ischaemia of the coronary arteries, which effect may be accentuated if there is torsion or kinking in the vessel as it is anastomosed, brought about by inaccurate selection of the location for fenestration on the graft or if the fenestration is made too large.

On the other hand, mobilisation or dissection of the proximal course of each coronary artery from surrounding tissues is not without risk as early branches may need to be divided, which may themselves bleed or worse still the main artery may be damaged inadvertently in the dissection which may cause bleeding and/or ischaemia. If such problems do occur then emergency coronary artery bypass grafting may be required, if the problem is recognised intra-operatively. If the problem is not recognised and dealt with promptly during the surgery, then the result of such coronary ischaemia can be fatal.

In the currently used technique of attachment of coronary artery buttons to aortic root replacement grafts, the surgeon must also use his judgment to select the site for making a hole or fenestration in the wall of the aortic root replacement graft. If this the selected site is either too high, too low or too much to the right or too much to the left then distance between the coronary button and the graft will greater and alignment imperfect, thereby predisposing to the problems outlined above.

To aid judgment of the site of the fenestration needs to be made, the surgeon may use techniques like temporarily distending the heart with blood to mimic the situation when the heart is full of blood and when the graft is full of blood and pressurised. However, this is only an approximation of the final position of the graft will take. So once again, careful judgement must be exercised by the surgeon in order to execute these steps effectively, otherwise, failure can be fatal for the patient.

As emphasised above, it is pertinent that many surgeries of this nature are performed in low numbers in many individual centres in the United States and other countries so experience of the individual surgeon may be limited and such limited experience may accentuate the risk of complications of the nature outlined above arising. Hence, any improvement that can eliminate or reduce the need reduce the need for judgement is likely to make the procedure safer, easier and faster to perform.

### Technique Replacement of the Aortic Root with Preservation of the Native Aortic Valve or Replacement of the Aortic Root with Replacement of the Aortic Valve

Where surgery entails replacement of the aortic root with replacement of the aortic valve, surgeons may either sew the valve to the graft and suture to the aortic annulus or many surgeons favour use of a composite valved graft wherein the graft and valve are supplied as a single unit or composite (assembled at the factory). Using a composite valved graft simplifies surgery because it only requires the valve end to be sewn to the aortic annulus as the graft has already been attached to the valve. Use of the composite valved graft also saves time and eliminates risk of bleeding at the junction between the graft and the valve. These grafts find favour with surgeons for their obvious advantages.

When surgery entails replacement of the aortic root with preservation of the native aortic valve, the valve leaflets are preserved during the surgical dissection along with a cuff of native aorta at the hinge point between the leaflet and aortic wall. Sutures are generally placed beneath the native aortic annulus to secure the lower end of the graft and cuff of native aorta or sinus subtending the base of each leaflet is attached inside of the graft with running sutures.

The coronary arteries are the first branches of the aorta as it leaves the heart. In both cases the coronary arteries have to be re-attached to the new graft. In general, a fenestration is made in the graft by the surgeon at an appropriate site and coronary artery anastomosed as a button with a running suture, working inside and outside of the graft. The precise location at which the surgeon makes the fenestration in the graft is down to surgical judgement. In general, the surgeon asks the perfusionist to obstruct venous return to the pump so that the heart distend or fills. This distension gives some better indication as to the optimal location of said fenestrations but selection of the precise location for each fenestration remains dependent on surgical judgement, even with tips and tricks, such as that outlined above and learnt from experience.

The diameter of the fenestration made by the surgeon for implantation of a currently available graft, is also down to surgical judgement. Experienced surgeons generally favour a small fenestration. However, less experienced surgeons sometimes adopt a larger fenestration to ease the subsequent anastomosis of the coronary button to the side wall of the graft at the fenestration but risking additional tension in the anastomosis which can be fatal.

### Bulged Portion of the Graft

The native aorta as it exits in the heart is characterised by having three bulges, commonly known in the field as the sinuses of Valsalva. These are thought to be important for normal opening and closure of the native aortic valve leaflets.

An advance in the field was therefore the inclusion of a bulge or expansion at the base of an aortic root replacement graft as shown in, European Patent application No. EP 0 955 019 A2 by Ruggero De Paulis entitled “Prosthetic tubular aortic conduit and method for manufacturing the same,” U.S. Pat. No. 8,388,679 by George Du entitled “Single continuous piece prosthetic tubular aortic conduit and method for manufacturing the same,” and U.S. Pat. No. 8,696,741 by George Du entitled “Woven prosthesis and method of manufacturing the same.”

Whilst the bulge described in the available patent documents was seemingly developed to provide a graft geometry closer to that of the native aortic root anatomy, with the presumption that said improvement would provide advantages to valve function analogous to those provided by natural sinuses of Valsalva. In addition, an unanticipated but significant additional benefit of this geometry observed by surgeons in the field, is that calibre of the graft being wider at this level makes implantation of the coronary ostia technically easier and less prone to complications that are known to be associated with this step in the operative procedure. However, it will be appreciated that a size discrepancy typically remains between the bulged portion of these grafts and the natural position of the coronary arteries, prior to mobilisation, in the diseased aortic root.

### Distal Anastomosis & Role of a Reinforcing Sleeve

Known problems at the distal anastomosis include bleeding and late pseudoaneurysm or false aneurysm formation.

Anastomotic pseudoaneurysm is a form of false aneurysm, whose wall does not consist of all normal layers of arterial wall. Given the rising number of reconstructive vascular procedures, the increase of anastomotic pseudoaneurysm cases is expected.

False aneurysms, also known as pseudoaneurysms, are abnormal outpouchings or dilatation of arteries which are bounded only by the tunica adventitia, the outermost layer of the arterial wall. These are distinguished from true aneurysms, which are bounded by all three layers of the arterial wall. Pseudoaneurysms typically occur when there is a breach in the vessel wall such that blood leaks through the inner wall but is contained by the adventitia or surrounding perivascular soft tissue.

A pseudoaneurysm, or pseudoaneurysm of the vessels, occurs when a blood vessel wall is injured and the leaking blood collects in the surrounding tissue. It is sometimes called a false aneurysm. In a true aneurysm, the artery or vessel weakens and bulges, sometimes forming a blood-filled sac.

In the ARR procedure, the surgeon makes two fenestrations in the aortic root replacement graft substitute and sews aorta surrounding the origins of the left and right coronary arteries to the graft.

Currently available ARR substitutes and methods of substitute ARR implantation procedures require that the surgeon either mobilise the coronary arteries so that the origins of the arteries may be drawn towards the graft and anastomosed without tension or stretch the arteries in order to bridge this gap and allow anastomosis of the coronary ostia to the grafts. Attendant risks of this approach are damage to the coronary arteries or early branches of the coronary arteries during mobilisation, inadvertent torsion of the coronary arteries during the anastomosis with attendant immediate risks of bleeding and/or ischaemia and residual tension at the suture line owing to inadequately judged extent of mobilisation with risk of early bleeding and/or late risk of false aneurysm formation at the suture line. These problems are accentuated in re-operations where tissues may be more difficult to mobilise.

During currently used methods of aortic root replacement using currently available grafts, a hole is made in the graft by the surgeon and a cannula or vent is inserted in the tubular portion of the graft and connected to the bypass machine so that residual air residing in the heart at the end of the procedure may be sucked or otherwise directed out of the circulation as the heart begins to eject. When little or no residual air is detected the venting cannula is removed and the hole created by the venting cannula repaired with sutures. It is not uncommon for the graft to bleed at this location and require compression and/or additional sutures to be placed. This adds time to the procedure and increases use of cardiotomy suction with deleterious effects on the circulation.

Thus, it is an object of the present disclosure to overcome one or more of the above-described drawbacks and/or disadvantages of the currently available grafts and systems.

### SUMMARY OF THE INVENTION

The present disclosure is directed toward grafts and methods for performing aortic root replacement surgery.

In one aspect of the present disclosure provided herein, is an aortic root replacement graft. The graft including a tubular portion; a bulged portion with a first end and a second end, wherein the second end of the bulged portion is coupled to a first end of the tubular portion; a cuff portion coupled to the first end of the bulged portion; and at least one side arm coupled to and extending from the bulged portion.

In another aspect of the present disclosure provided herein, is method for performing aortic root replacement surgery. The method includes preparing a patient for open heart surgery and obtaining the graft. The method also includes coupling a second end of the graft to a distal aorta and coupling a first end of the graft to at least one of a proximal aorta and an aortic annulus. In addition, the method includes coupling a first side arm to a first coronary artery and coupling a second side arm to a second coronary artery. Finally, the method includes completing the open heart surgery and closing the patient.

These and other objects, features and advantages of this disclosure will become apparent from the following detailed description of the various aspects of the disclosure taken in conjunction with the accompanying drawings.

### BRIEF DESCRIPTION OF THE DRAWINGS

The accompanying drawings, which are incorporated in and constitute a part of the specification, illustrate embodiments of the disclosure and together with the detailed description herein, serve to explain the principles of the disclosure. It is emphasized that, in accordance with the standard practice in the industry, various features are not drawn to scale. In fact, the dimensions of the various features may be arbitrarily increased or reduced for clarity of discussion. The drawings are only for purposes of illustrating preferred embodiments and are not to be construed as limiting the disclosure.

FIG. 1 is a perspective view of an aortic root replacement graft, in accordance with an aspect of the present disclosure;

FIG. 2 is a front view of the aortic root replacement graft of FIG. 1, in accordance with an aspect of the present disclosure;

FIG. 3 is a rear view of the aortic root replacement graft of FIG. 1, in accordance with an aspect of the present disclosure;

FIG. 4 is a left side view of the aortic root replacement graft of FIG. 1, in accordance with an aspect of the present disclosure;

FIG. 5 is a right side view of the aortic root replacement graft of FIG. 1, in accordance with an aspect of the present disclosure;

FIG. 6 is a top view of the aortic root replacement graft of FIG. 1, in accordance with an aspect of the present disclosure;

FIG. 7 is a bottom view of the aortic root replacement graft of FIG. 1, in accordance with an aspect of the present disclosure;

FIG. 8 is a top view of a heart with a dilated native ascending aorta showing the typical morphology of a dilated aorta in need of aortic root replacement surgery, in accordance with an aspect of the present disclosure;

FIG. 9 is a cross-sectional view through the chest of a patient with the dilated ascending aorta of FIG. 8, in accordance with an aspect of the present disclosure;

FIG. 10 is another view of the ascending aorta and heart of FIG. 8 with ribs and the brachiocephalic artery from the aorta distally, in accordance with an aspect of the present disclosure;

FIG. 11 is a cross-sectional view of the dilated ascending aorta of FIGS. 8-10 showing the origins of the left coronary artery (LCA) and right coronary artery (RCA) having a dilated native aorta with a radius of R1, in accordance with an aspect of the present disclosure;

FIG. 12 is a cross-sectional view of the dilated native aorta of FIG. 11 showing a typical aortic root replacement graft superimposed in dotted lines upon the dilated native aorta with the tubular portion of a typical aortic root replacement graft having a radius of R2 and the bulged portion of a typical aortic root replacement graft having a radius of R3, in accordance with an aspect of the present disclosure;

FIG. 13 is a top view of the heart of FIGS. 8-11 after implantation of a typical aortic root replacement graft, in accordance with an aspect of the present disclosure;

FIG. 14 is another view of the ascending aorta and heart after implantation of the aortic root replacement graft of FIGS. 1-7, in accordance with an aspect of the present disclosure;

FIG. 15 is a magnified view of the aortic root replacement graft in FIG. 14 showing the anastomosis of the LCA and RCA to the side arms of the graft, the distal anastomosis between the tubular portion of the graft and the native distal aorta, and the proximal anastomosis between the graft and the native aortic annulus, in accordance with an aspect of the present disclosure;

FIG. 16 is the magnified view of FIG. 15 further showing the tubular portion of the graft, the bulged portion of the graft, a side arm on the bulged portion, the venting chimney, the reinforcing sleeve, and the aortic valve prosthesis attached at the proximal end of the graft, in accordance with an aspect of the present disclosure;

FIG. 17 is a magnified view of the aortic root replacement graft of FIG. 16 with the venting chimney tied after the operation is completed and venting is no longer required and the reinforcing sleeve equipped with a suture that is tightened around the proximal side of the distal anastomosis, in accordance with an aspect of the present disclosure;

FIG. 18 is a magnified view of the aortic root replacement graft of FIG. 16 including a reinforcing sleeve secured on both the distal and proximal sides of the distal anastomosis and the anastomosis of the side arms to the coronary arteries (LCA and RCA), in accordance with an aspect of the present disclosure; and

FIG. 19 is a perspective view of the aortic root replacement graft of FIG. 1, in accordance with an aspect of the present disclosure.

### DETAILED DESCRIPTION FOR CARRYING OUT THE INVENTION

Generally stated, disclosed herein are an aortic root replacement graft and methods for performing aortic root replacement surgery.

In this detailed description and the following claims, the words proximal, distal, anterior, posterior, medial, lateral, superior, and inferior are defined by their standard usage for indicating a particular part or portion of an organ, instrument, or implant according to the relative disposition of the natural bone or directional terms of reference. For example, “proximal” means the portion of a device or implant nearest the torso, while “distal” indicates the portion of the device or implant farthest from the torso. As for directional terms, “anterior” is a direction towards the front side of the body, “posterior” means a direction towards the back side of the body, “medial” means towards the midline of the body, “lateral” is a direction towards the sides or away from the midline of the body, “superior” means a direction above and “inferior” means a direction below another object or structure.

Similarly, positions or directions may be used herein with reference to anatomical structures or surfaces. For example, as the current grafts, systems, instrumentation, and methods are described herein with reference to use with the aortic root, the heart may be used to describe the surfaces, positions, directions or orientations of the grafts, systems, instrumentation, and methods. Further, the grafts, systems, instrumentation and methods, and the aspects, components, features and the like thereof, disclosed herein are described with respect to the thoracic cavity for brevity purposes, but it should be understood that the grafts, systems, instrumentation, and methods may be used with other arteries of the body having similar structures.

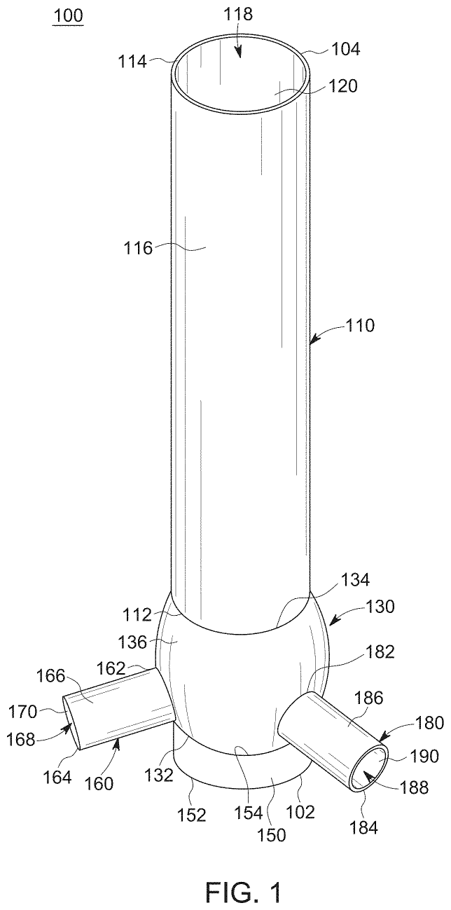

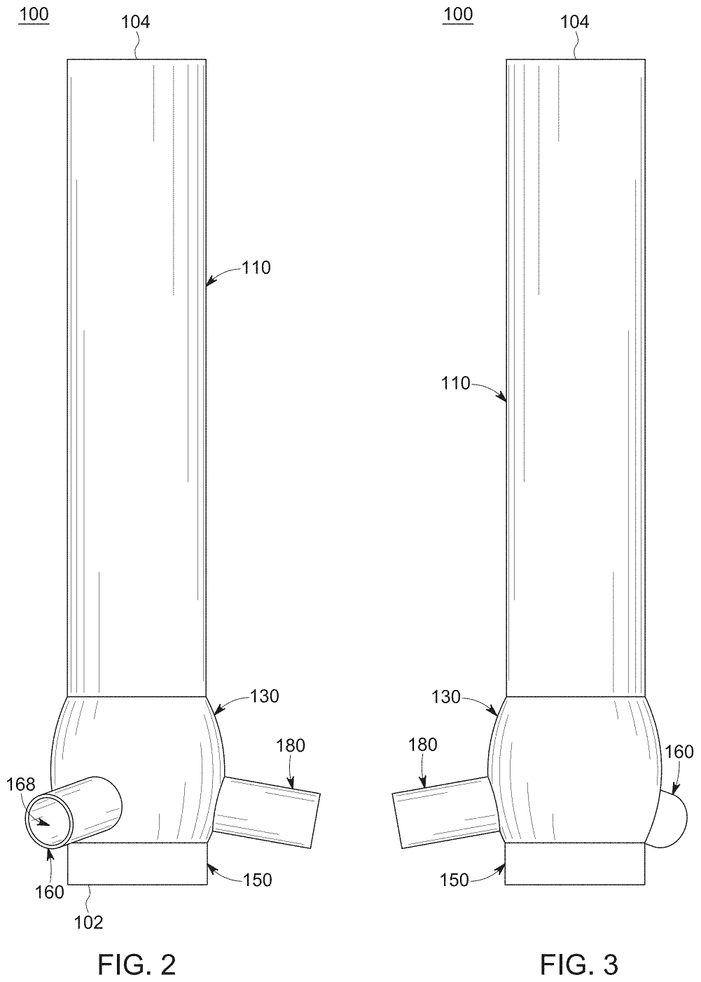

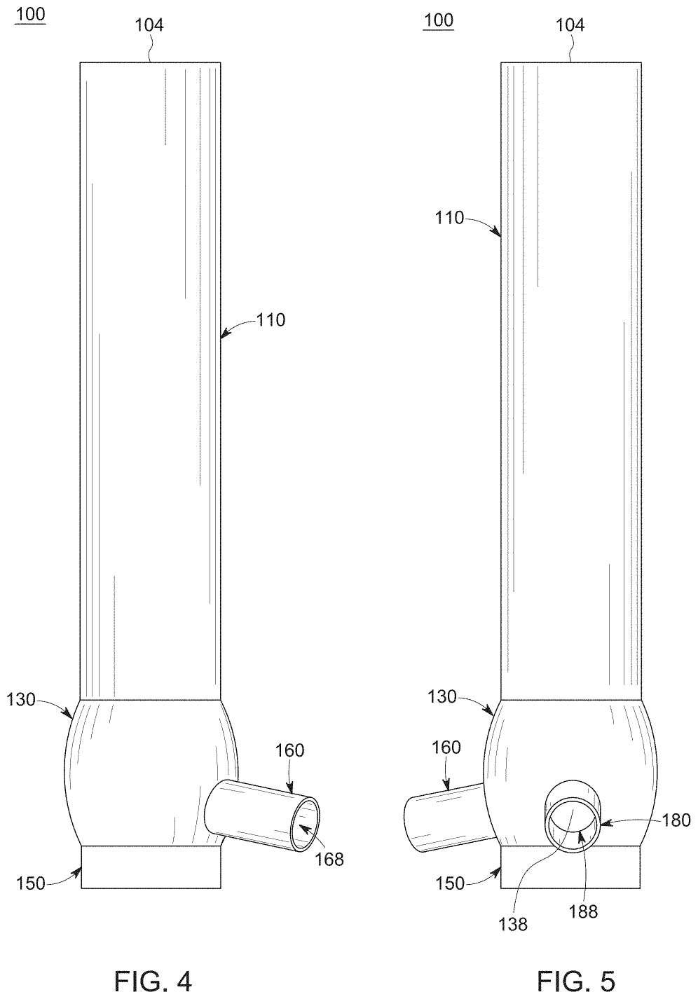

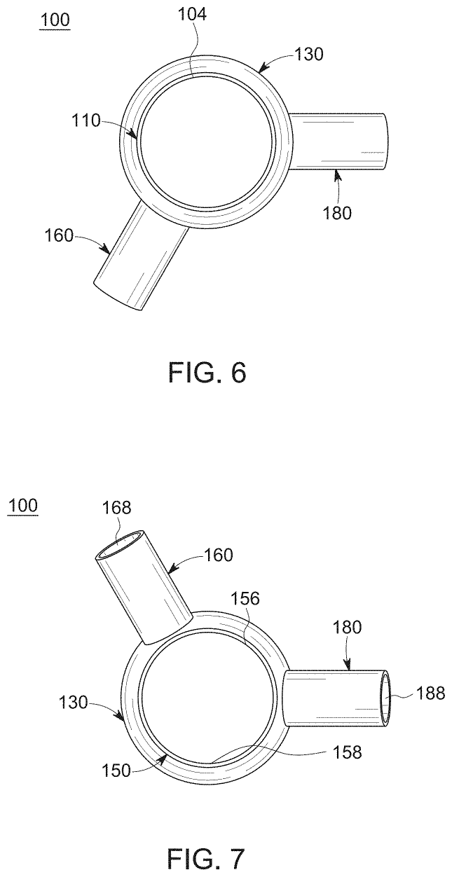

Referring to the drawings, wherein like reference numerals are used to indicate like or analogous components throughout the several views, and with particular reference to FIGS. 1-7 and 14-18, the graft and methods for performing aortic root replacement surgery are shown.

Referring now to FIGS. 1-7 and 14-18, the aortic root replacement (ARR) graft 100 is shown. The graft 100 includes a first or proximal end 102 and a second or distal end 104. The graft 100 also includes a tubular portion 110, a bulged portion 130, a cuff portion 150, and at least one arm or tube 160, 180. The tubular portion 110 extends from the second end 104 toward the first end 102 and couples to the bulged portion 130. The bulged portion 130 couples to the tubular portion 110 on one end and to the cuff portion 150 on another end. The cuff portion 150 couples to the bulged portion 130 on one end and extends to the first end 102 of the graft 100. The at least one arm 160, 180 is coupled to and extends away from the bulged portion 130. The at least one arm 160, 180 bridge the gap between the bulged portion 130 of the graft 100 and the surrounding anatomical structures containing the coronary arteries. The diameter of the bulged portion 130 is, for example, larger than the diameters of the tubular portion 110 and the cuff portion 150.

The bulged portion 130 of Valsalva includes side arms 160, 180 to overcome size discrepancy between the bulged portion 130 of the graft 100 and the location of the native coronary arteries. The graft 100 also incorporates a venting chimney 210 for efficient venting of air at the conclusion of the operation. In addition, the graft 100 incorporates a reinforcing sleeve 200 to support the distal anastomosis between the ARR graft 100 and native aorta 230 for a faster and more secure haemostasis at the end of the procedure and to mitigate against the risk of late false aneurysm formation at this suture line. Finally, the graft 100 may be used with a suitable sizer. The graft 100 may be, for example, a singular integral or monolithic piece (i.e., of one-piece construction), or may be formed from a plurality of components that are coupled together to form the graft 100.

With continued reference to FIGS. 1-7, the tubular portion 110 includes a first or proximal end 112 opposite a second or distal end 114. The tubular portion 110 also includes an exterior surface 116 extending between the first end 112 and the second end 114. In addition, the tubular portion 110 includes a through hole 118 extending through the length of the tubular portion 110 from the first end 112 to the second end 114. The through hole 118 is formed by an interior surface 120 of the tubular portion 110. The through hole 118 provides for fluid communication between the distal portion of the aorta 230 and the bulge portion 130 of the graft 100. The tubular portion 110 may also include a venting chimney 210 coupled to and extending away from the exterior surface 116 of the tubular portion 110 between the first end 112 and the second end 114.

As shown in FIGS. 1-7, the bulged portion 130 includes a first or proximal end 132 opposite a second or distal end 134. The bulged portion 130 also includes an exterior surface 136 positioned on an outside of the bulged portion 130 and an interior surface 138 positioned on an interior of the bulged portion 130. The interior surface 138 of the bulged portion 130 is in fluid communication with the interior surface 120 of the tubular portion 110 to allow for blood flow between the distal portion of the aorta 230 and the proximal portion of the aorta 230 and heart 220. The first end 132 of the bulged portion 130 couples to the second end 154 of the proximal cuff portion 150. In addition, the second end 134 of the bulged portion 130 couples to the first end 112 of the tubular portion 110.

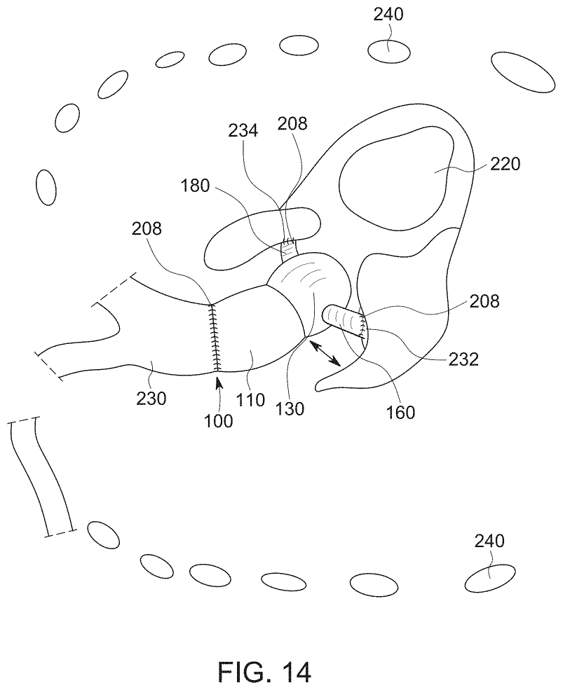

The cuff portion 150 is positioned for engagement with the proximal portion of the aorta 230, as shown in FIGS. 14-18. With continued reference to FIGS. 14-18 and reference to FIGS. 1-7, the cuff portion 150 includes a first or proximal end 152 opposite a second or distal end 154. In addition, the cuff portion 150 includes an exterior surface 156 positioned on an outside of the cuff portion 150 and an interior surface 158 positioned on an interior of the cuff portion 150. The interior surface 158 of the cuff portion 150 is in fluid communication with the interior surface 138 of the bulged portion 130 to allow for blood flow between the proximal portion of the aorta 230 and heart 220 and the distal portion of the aorta 230.

The at least one arm 160, 180 may be, for example, a first arm or tube 160 and a second arm or tube 180. The first arm 160 may couple to a right coronary artery 232 and the second arm 180 may couple to a left coronary artery 234, as shown in FIGS. 14-18. The first arm 160 includes a first end 162 coupled to the bulged portion 130 and a second end 164 configured to or for coupling to a right coronary artery 232. The first arm 160 also has an exterior surface 166 positioned on an outside of the arm 160 and extending from the first end 162 to the second end 164. The first arm 160 also includes a first opening 168 extending through the first arm 160 along a longitudinal axis between the first end 162 and the second end 164. The first opening 168 is formed by an interior surface 170 extending between the first end 162 and the second end 164 and allowing for fluid communication between the interior surface 138 of the bulged portion 130 and a right coronary artery 232.

The second arm 180 includes a first end 182 coupled to the bulged portion 130 and a second end 184 configured to or for coupling to a left coronary artery 234. The second arm 180 also has an exterior surface 186 positioned on an outside of the arm 180 and extending from the first end 182 to the second end 184. The second arm 180 also includes a second opening 188 extending through the second arm 180 along a longitudinal axis between the first end 182 and the second end 184. The second opening 188 is formed by an interior surface 190 extending between the first end 182 and the second end 184 and allowing for fluid communication between the interior surface 138 of the bulged portion 130 and a left coronary artery 234.

With continued reference to FIGS. 1-7, the arms 160, 180 are pre-attached to the bulged portion 130 to prevent anastomosis that occurs with currently available grafts when coronary buttons are used. The arms 160, 180 are coupled to the bulged portion 130 to allow for attachment of the coronaries at their location and the graft end 102 brought to the coronary artery rather than vice versa. The side arms 160, 180 remove the need for (1) mobilisation of coronary arteries and (2) the surgeon to select a site for fenestration of a graft.

The side arms 160, 180 are moveable and can be bent to the left or to the right and up or down. The side arms 160, 180 may also be stretched without applying significant tension at each end. These properties of the side arms 160, 180 may be conferred by the choice of material itself or by the use of crimping of said materials in the manufacturing process. Thus, the side arms 160, 180 are able to be anastomosed to a coronary button without the need to identify a site for a fenestration in a graft and without the need to mobilise the coronary buttons.

The side arms 160, 180 are attached to the aortic root replacement graft 100 and may be directed in a generally downward direction, that is angled towards the base or aortic valve end 102 of the graft 100, as shown in FIGS. 1-5. Angling the side arms 160, 180 in a downward direction allows for the engagement with the native coronary arteries, which course into the right or left atrioventricular groove that is in a generally downward direction. In addition, since the coronary arteries may arise at different ‘heights’ relative to the plane of the native aortic valve annulus, in particular in relation to the nadir of the hinge points of the base of the leaflets, having the side arms 160, 180 capable of being bent up or down means that a given design of graft 100 with pre-set position of side arms 160, 180 will accommodate differences in height of the coronary arteries between individual patients without the need for additional sets of grafts with different heights of side arms 160, 180.

Further, the side arms 160, 180 allow for the anastomosis to be positioned between the graft 100 and the coronary buttons which is end-to-end. The side arms 160, 180 carry the anastomosis away from the main body or bulged, sinus portion 130 of the graft 100 so that the anastomosis cannot distort the sinus or main body 130 of the graft 100 in any way that might distort or interfere with function of the heart valve. In addition, the side arms 160, 180 allow for the entire anastomosis to be done from outside of the graft 100 so the graft 100 does not need to be trimmed to length for the distal anastomosis until later in the operation and moreover, any length of graft 100 can be maintained for replacement of the entire ascending aorta and even part or all of the aortic arch without interruption as the surgeon does not need to operate inside the graft 100 to anastomose the coronary buttons.

With continued reference to FIGS. 1-7 and 14-18, the side arms 160, 180 may have variable lengths and variable diameters. The side arms 160, 180 may have sizes of, for example, approximately 8 mm-10 mm, or more specifically, may be 8 mm or 10 mm for construction of end-to-end anastomosis in the method of performing aortic root replacement surgery with the graft 100, however, other sizes of side arms 160, 180 are also contemplated. The resting length of the side arms 160, 180 of the aortic root replacement graft 100 may be, for example, approximately 15 mm, more specifically 15 mm, which can be cut to appropriate length. Other lengths of the side arms 160, 180 are also contemplated. In addition, the side arms 160, 180 may be disposed, for example, approximately 115 degrees to 185 degrees, more specifically 120 degrees or 180 degrees relative to one another about the bulged portion 130. Further, the side arms 160, 180 may be disposed in an outward direction and/or a downward direction as the arms 160, 180 extend from the bulged portion 130. The downward direction of the arms 160, 180 may be the result of the arms 160, 180 coupling to the bulge portion 130 of the graft 100 either perpendicular to the bulge portion 130 below the centre line or bevelled to point downwards. The arms 160, 180 are positioned in this orientation based on the native annulus being crown shaped in its geometry. The amplitude of the crown may be modified by the pathology or exhibit little modification and be very large in amplitude. Composite valved conduits tend to draw all of the annular tissues into one plane, a new plane, not easily predictable as it depends upon how much the tissue rise up to meet the composite.

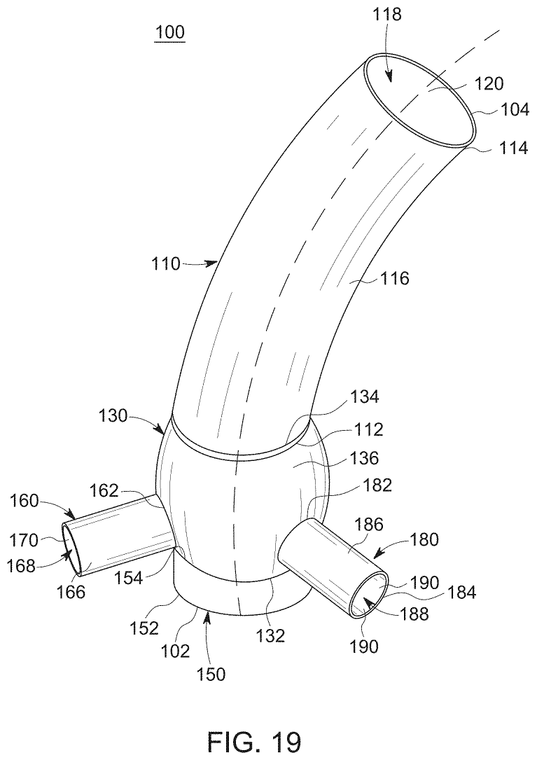

The side arms 160, 180 may have flexibility to bend upwards or downwards, to the left or to the right and accept some degree of torsion removes the need for careful judgement and expertise. The flexibility of the side arms 160, 180 may be provided by making the side arms 160, 180 corrugated or crimped in structure. Further, the structure or texture of the grafts 100 may provide a degree of elasticity or flexibility so that the arms 160, 180 can bend without being kinked. As kinking might disrupt the flow of blood and precipitate clot formation which might otherwise embolise causing end organ ischaemia such as myocardial infarction, stroke, mesenteric infarction etc. depending on the vascular bed in which the clot becomes lodged. As shown in FIG. 19, the tubular portion 110 or a tube, including the tubular portion 110, the bulged portion 130, and the cuff portion 150, may have a natural curvature to its long axis, mimicking the natural curvature of the native aorta, to favour less kinking following implantation.

The side arms 160, 180 can be trimmed to shorten their length by the surgeon during the operation. The appropriate length may be guided by measurement of the pre-operative computed tomography (CT) or assessment of length at the time of surgery. Measuring the distance from the origins of the native coronary arteries to the projected centreline of the aortic root replacement graft 100 may be performed on computed tomography (CT) with or without additional software or the distance between the origins of the two coronary arteries may be measured to guide trimming of the side arms 160, 180 during the operative procedure.

The side arms 160, 180 are attached at approximately 90 degrees to the surface of the fabric but the location of said attachment is located below the maximum radius of the bulge portion 130 in the graft 100 so that the long axis of the side arms 160, 180 effectively points downwards i.e., towards the base 150 of the graft 100. This is important because the natural direction of the coronary arteries is away from the graft 100 in a lateral and downward direction. Also, the implantation of the graft substitute 100 at the aortic annular level is such that the graft 100 has a tendency to sit high hence the side arms 160, 180 need to point downwards. The location of the side arms 160, 180 on the graft 100 and their downward direction renders the actual anastomosis between the side arm 160, 180 and coronary artery 232, 234 more accessible for inspection and/or repair with additional sutures at the end of the procedure.

The side arms 160, 180 are preferably made from a fabric that is similar to the material used for the main body 110 of the aortic root replacement graft 100 and moreover, crimped as this reduces the likelihood of the side arm 160, 180 being kinked or under tension or under torsion at the end of the anastomosis, once again rendering surgery much easier and the final result less subject to eyeballing and judgement of the individual surgeon.

The side arms 160, 180 may also have one or more markers, such as coloured line or lines, disposed along the length of the graft 100 and visible on the external surface 116, 136 of the graft 100 to aid orientation and spacing of sutures during the anastomosis of the coronary button to aid faster and more accurate placement of sutures during surgery. Markers may divide the circumference of the side arms 160, 180 into quadrants or thirds for ease of anastomosis, as discussed herein.

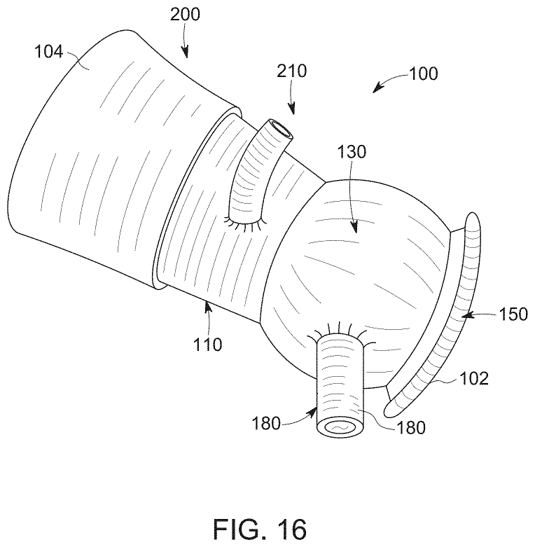

Referring now to FIGS. 16-18, a reinforcing sleeve 200 which may be used with the graft 100 is shown. A reinforcing sleeve or external supporting structure 200 can reduce or eliminate bleeding from the distal anastomosis and late false aneurysm formation by positioning the structure 200 neatly over the distal anastomosis of the graft 100. The reinforcing sleeve 200 is designed and sized so that it may be telescoped over the aortic root replacement portion 110 of the graft 100 to provide external support for the distal anastomosis. The reinforcing sleeve 200 may also create a clean and tidy distal anastomosis, reduce or eliminate bleeding from the anastomosis, prevent or reduce in the frequency of formation of false aneurysm at the anastomotic site, and prevent or reduce in tendency of aorta to dilate further over the extent of the external covering. In addition, the native aorta is commonly, but not always, larger in diameter than the replacement graft 100. The diameter of the distal side of the distal anastomosis may be rendered even larger if reinforcing material like Teflon felt has been incorporated into the distal suture line.

As shown in FIG. 16, the reinforcing sleeve 200 is for reinforcement of the distal anastomosis. In its simplest form the reinforcing sleeve 200 is a straight tube whose internal diameter is of sufficient calibre that its internal surface sits snugly over the external surface of the tubular portion 110 of the aortic root replacement graft 100.

The reinforcing sleeve 200 has proximal and distal portions. Proximal and distal portions may be of the same or different radius. Preferably the radius of the distal portion is larger than the radius of the proximal portion in order to accommodate the larger radius of the native distal aorta 230 at the anastomosis which is most commonly found during aortic root replacement operations. The distal portion is designed to be drawn around the distal side of the graft 100 to native aorta 230 anastomosis and the proximal portion is designed to be drawn around the graft 100 side of the anastomosis. The encircling ligatures or drawstrings 250 are disposed on the portion of larger radius by which to tighten the reinforcing sleeve 200 around the distal (native aortic) side of the distal anastomosis. In addition, the encircling ligatures or drawstrings 250 are disposed on the portion of the reinforcing sleeve 200 with smaller radius by which tighten the reinforcing sleeve 200 around the proximal (graft) side of the distal anastomosis.

The reinforcing sleeve 200 may be, for example, wider at one end, i.e., have a flared portion to accommodate the larger aorta with or without additional reinforcing material. The inner diameter of the tubular portion 110 of the reinforcing sleeve 200 is larger than the diameter of the external surface 116 of the main body or tubular portion 110 of the aortic root replacement graft 100. The larger diameter of the reinforcing sleeve 200 allows for the reinforcing sleeve 200 to slide like a telescope over the tubular portion 110 of the graft 100. The reinforcing sleeve 200 may be either straight or flared portion so as to fit over distal aorta. The reinforcing sleeve 200 may include an accompanying material incorporated into the suture line anastomosis, such as pericardium or Teflon felt.

The sleeve 200 is preferably provided with one or more pre-loaded drawstrings 250 on proximal and distal portions, and with one or more drawstrings 250 on each of the proximal and distal portions. The reinforcing sleeve 200 is placed around the distal anastomosis between the aortic root replacement graft 100 and the native distal aorta 230. Reinforcement has dual purposes of providing external support to reduce the possibility of late aneurysm formation and also to control bleeding at the distal anastomosis and promote haemostasis at the end of the surgical procedure.

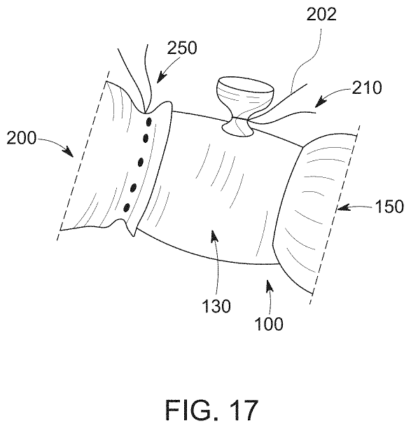

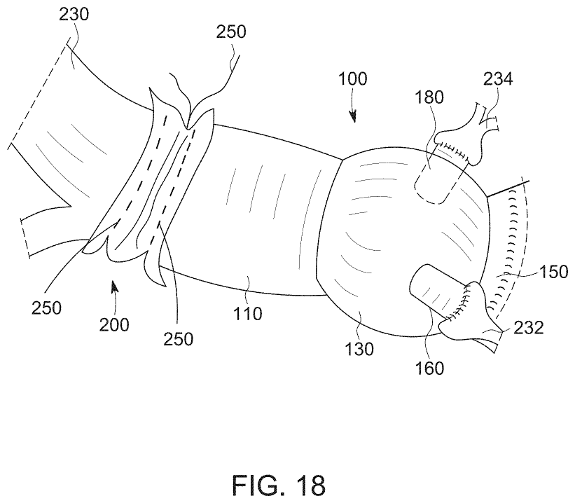

As shown in FIGS. 17-18, the reinforcing sleeve 200 may be provided with a drawstring 250 passed around, or indeed interweaved as a purse string around each end of the reinforcing sleeve 200 so that the sleeve 200 may be tightened around the distal anastomosis so as to create a seal at the anastomosis. Alternatively, reinforcing sleeve 200 may be marked so that the surgeon can place the drawstring 250. More preferably the reinforcing sleeve 200 is supplied with drawstrings 250 with the obvious benefit that this saves time. Moreover, multiple bites make for more even closure around the graft 100 and being more drawstring 250 accentuates the benefits of having this done by the manufacturer.

In addition, the portion of the reinforcing sleeve 200 that is positioned over the distal aorta may also have suture 202. The reinforcing sleeve 200 may also include more than one suture line 202 to spread the load over aorta. The reinforcing sleeve 200 may also include a cuff of cushioning material, like Teflon felt. The sleeve structure 200 may be placed over the distal stump of the aortic root replacement graft 100 prior to anastomosis and then pulled up over the anastomosis. However, if the native aorta is short and space is limited or if the surgeon inadvertently neglects this step, then the sleeve structure 200 may be incised between the two suture ends exit the graft material and passed around the distal anastomosis and tied as above.

It is generally easier in surgery to perform an end-to-end anastomosis. Ease of performing anastomosis accurately and neatly and speedily may be improved by having additional marks on the graft 100 signifying division into quadrants or thirds. Therefore, the aortic root replacement graft 100 may be equipped with markings signifying division into quadrants or thirds. Said markings may be on one or both ends of the graft, on the side arms and/or on either or both ends of the reinforcing sleeve.

In an embodiment, the reinforcing sleeve 200 may be loaded onto the graft 100 prior to performing the distal anastomosis. In this case the sleeve 200 would then be drawn over the distal anastomosis upon completion and drawstrings 250 tied, as shown in FIGS. 17-18. Alternatively, the surgeon may cut the reinforcing sleeve 200 between the drawstring ends 250 and pass the reinforcing cuff around the distal anastomosis after completion and the tighten the drawstrings 250 as above.

The drawstrings 250 may either be tightened temporarily with a tourniquet or permanently tightened by tying. The advantage of temporarily tightening drawstrings 250 with a tourniquet is that the tourniquet may be loosened at intervals so that the distal anastomosis may be re-inspected to confirm adequate haemostasis prior to permanent tightening and closure of the patient at the very end of the surgical procedure. Once haemostasis is confirmed the drawstrings 250 may then be permanently drawn around the anastomosis by tying. In addition, by temporarily tightening the drawstrings 250 with a tourniquet the haemostatic agents may be injected or placed between the reinforcing sleeve 200 and the reinforcing cuff should haemostasis prove challenging prior to the drawstrings 250 being permanently tightened. Further by temporarily tightening drawstrings 250 with a tourniquet additional interrupted sutures may be placed in the distal anastomosis prior to the drawstrings 250 being permanently tightened.

A line marking the portion of the reinforcing graft 100 that lies between the two ends of the respective drawstrings 250, as shown in FIG. 18, may be marked on the graft 100 to guide the surgeon in cutting the reinforcing sleeve 200 so that he does not inadvertently cut one or more of the drawstrings 250.

With continued reference to FIGS. 16-18, the reinforcing sleeve 200 may be made from a similar material to the aortic root replacement graft 100 or alternatively may be made from a material, such as Teflon felt, with more compressive properties that has the advantage of that the reinforcing sleeve 200 may be gently squeezed over the distal anastomosis to improve haemostasis. Alternatively, the reinforcing sleeve 200 may be made from similar material to the aortic root replacement graft and incorporate a reinforcing strip of compressible material, such as Teflon felt, on its proximal or distal portions for compression effect when drawstrings 250 are tightened and improved haemostasis. Occasionally the situation is reversed and in such situations the reinforcing sleeve 200 may be flipped around.

When the reinforcing sleeve 200 is loaded onto the graft 100 prior to performing the distal anastomosis, one or more temporary encircling ligatures may be passed around the reinforcing sleeve 200 to reduce its length so that it does not get in the way of the surgeon whilst he is performing the distal anastomosis. The temporary encircling ligatures may be cut and removed so that the reinforcing sleeve 200 may be drawn over the distal anastomosis upon completion. Temporary encircling ligatures or drawstrings 200 may be pre-assembled on the reinforcing sleeve during manufacture or performed by the surgeon during surgery.

When the reinforcing sleeve 200 has been cut in a longitudinal direction the two edges may be closed with running or interrupted sutures or else left open at the surgeon's discretion.

In order to facilitate temporary or permanent tightening of the drawstrings 250 the pressure in the circulation may be temporarily reduced by known methods including but not limited to pharmacological means or by temporarily reducing flow from the cardiopulmonary bypass rapid ventricular pacing or by placing the patient in Trendelenburg position. Pressure may then be restored once drawstrings 250 have been tightened.

One or more sutures may optionally be placed through the reinforcing sleeve 200 and at least one of the aorta 230, the aortic root replacement graft 100, any reinforcing material used to perform the distal anastomosis to prevent migration of the reinforcing sleeve 200.

Alternatively, the drawstrings 250 passed around entire graft 100 may be replaced with sutures 208 at the two ends, which may be temporarily or permanently tied, as described above. The reinforcing sleeve 200 may either be provided with said drawstring sutures 250, or these may be placed by the surgeon at the time of surgery.

The radius of the proximal portion of reinforcing sleeve 200 is preferably adapted so that it slides snugly over the tubular portion 110 of the aortic root replacement graft 100. In other words, the internal diameter of the proximal portion of the reinforcing sleeve 200 should preferably be similar to the external diameter of the tubular portion 110 of the aortic root replacement graft 100, within known tolerances for handling characteristics and techniques for manufacture for grafts 100 made from such materials.

Also shown in FIGS. 16-17, the graft 100 may include a side arm or venting chimney 210 coupled to and extending away from the tubular portion 110. In an embodiment, the side arm 210 may be for the brachiocephalic artery or bovine trunk. The side arm 210 of the aortic root replacement graft 100 may have a suitable diameter for anastomosis to a brachiocephalic trunk or more particularly a bovine trunk. The side arm 210 may also be disposed at an angle to the central axis of the graft 100 for anastomosis to a brachiocephalic trunk or more particularly a bovine trunk, where the aortic aneurysm is of such an extent that dilatation includes the origin of said brachiocephalic artery or bovine trunk.

In another embodiment, such as shown in FIG. 16, the side arm 210 may be configured as a venting chimney 210. The venting chimney 210 may be a tube graft attached during manufacture. The attachment is preferably on the straight portion of the tube graft 110 and preferably at approximately 90 degrees to the fabric surface. By having a venting chimney 210 attached to the graft 100 during manufacture, it is simple and quick for the surgeon to connect the venting chimney 210 directly with the venting tubing for venting of air. When little or no residual air is detected the venting chimney 210 may simply be tied off with a tie or suture 202, as illustrated in FIG. 17, without the need for sutures penetrating the venting chimney 210 and with little or no risk of bleeding.

The venting chimney 210 should preferably be oriented so that it generally directed upwards from the uppermost portion of the graft 100 following implantation. The left coronary ostium is taken as a reference point and the venting chimney 210 should be positioned at a radial position of 120 degrees around the central axis of the graft 100 from the side arm 180 for the left coronary anastomosis 234 and at similar radial position to the central axis as the side arm 160 for the right coronary artery 232. In other words, the venting chimney 210 is disposed so that it arises at a radial position approximately 120 degrees in relation to the radial position of the left coronary side arm 180 to the long axis of the tubular portion 110 of the graft 100. In addition, the venting chimney 210 should preferably be positioned in the tubular portion 110 of the aortic root replacement graft 100.

The calibre of the venting chimney 210 should preferably be of sufficient size to connect directly with a ¼ inch connector typically found on venting lines in cardiopulmonary bypass circuits. However, venting chimney 210 larger or smaller in calibre are also possible and contemplated for use with the graft 100.

Although not shown, the graft 100 of FIG. 18 may also include a venting chimney 210.

In another embodiment, the graft 100 may include a valve prosthesis (not shown). The aortic root replacement graft 100 may include a valve prosthesis attached using known methods of attachment, so as to provide a ‘pre-assembled valved conduit’ version of the device 100 that may be implanted using the methods discussed in greater detail below. The valve prosthesis may be a mechanical valve, a biological valve or a polymeric valve, or other type of valve structure. A mechanical valve may have at least one leaflet, for example, one leaflet, two leaflets, or a three leaflet morphology. The valve may be attached to the end of the graft 100 just at or below the bulged portion 130 with or without trimming of the collar portion or cuff portion 150.

In yet another embodiment, the graft 100 may include a sizer (not shown) for the aortic root replacement graft 100 with side arms 160, 180 that incorporates the valve size of the graft 100. The sizer may also incorporate the radial position and height of the preformed side branches 160, 180 in relation to the sewing cuff of a pre-assembled aortic valve or aortic graft composite so that the surgeon can choose a graft 100 of the correct size and position the graft 100 using the side branches 160, 180 as the reference to the point of rotation.

In addition, the sizer for the aortic root replacement graft 100 may include pre-formed coronary buttons that accurately reflect the height, length, and radial disposition of the side arms 160, 180 of the graft 100. The sizer may be used to determine the diameter of required graft 100. A lower portion of the sizer may be generally circular in form or cylindrical for sizing the aortic annulus. This lower portion of the sizer may match the profile of a valved conduit composite more closely than a simple cylinder. The sizer may also be used to determine orientation of implantation of the graft 100 so the sizer may have marks corresponding to one or more valve commissures as references points and corresponding with the position of markers on the valve graft composite. As above with the markers on the actual valve-graft composite may be disposed in equal thirds around the circumference of said composite. The sizer may have a handle disposed along longitudinal axis for manipulating the sizer and holding it in position. The handle may be, for example, straight, curved or angled in profile to aid manipulation. Finally, the sizer may be disposed with one or more lights to aid visualisation during sizing, this being especially beneficial if used in minimally invasive surgery where provision of light may be limited from external light sources that are obscured by the margins of small sized incision or incisions.

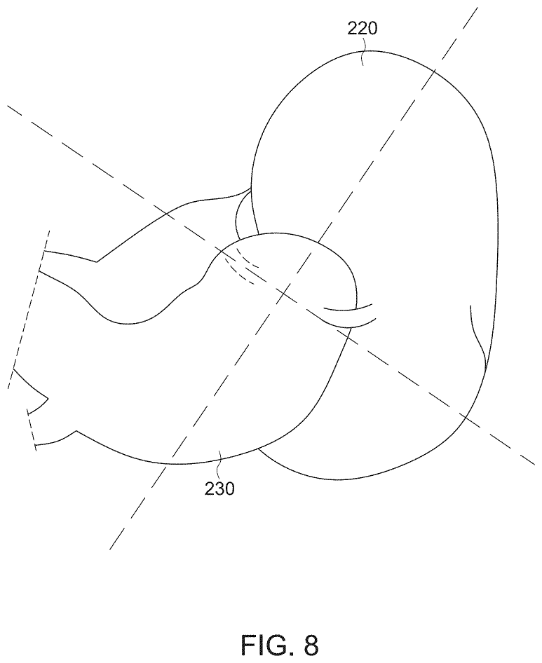

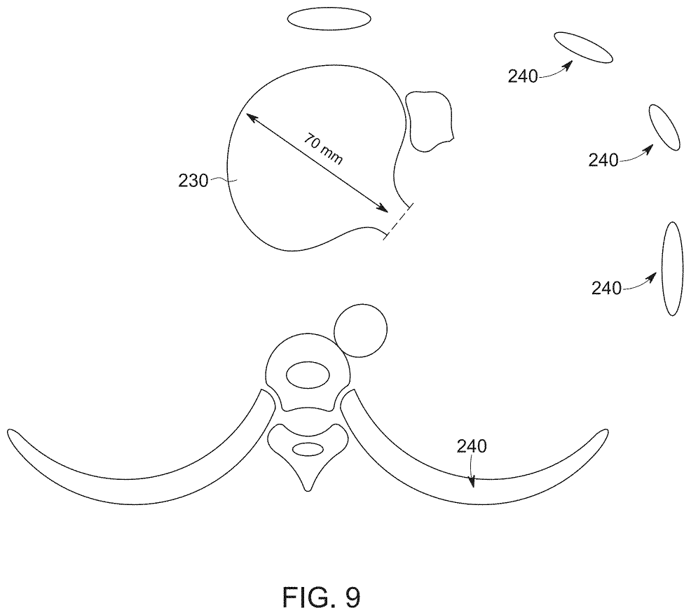

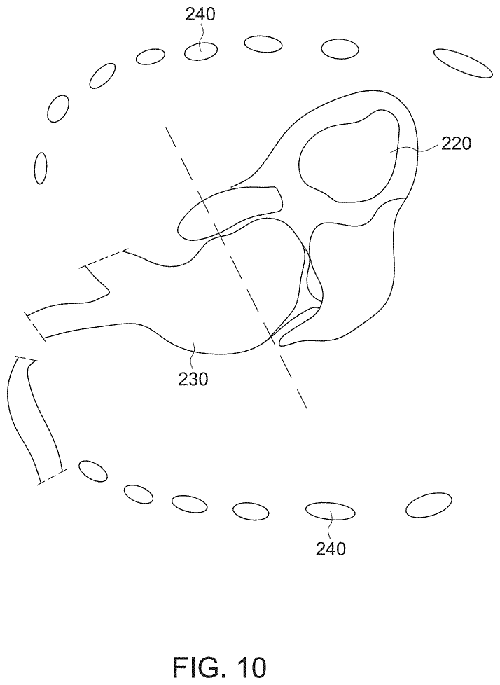

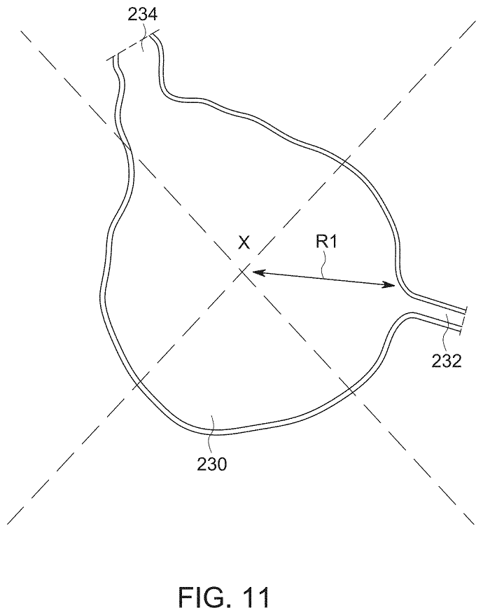

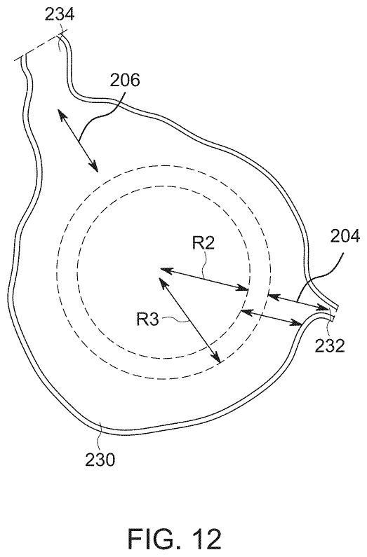

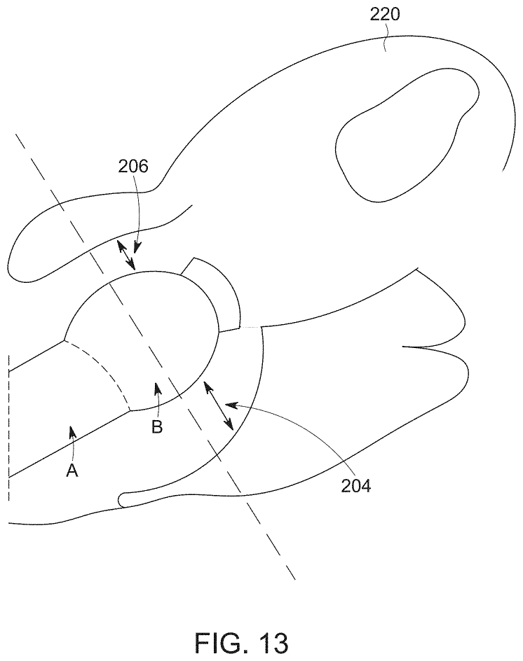

Referring now to FIG. 8, the anatomy of a patient presenting with an aneurysm of the ascending aorta and aortic root is shown. In addition, FIGS. 9-10 show the aorta 230 and heart 220 inside the chest. With reference to FIG. 11, a cross-section of the aorta 230 showing the origins of the left and right coronary arteries 232, 234 is shown. Further, FIG. 9 shows a cross-section through an aorta 230 affected by aneurysmal dilation, at the level of the origins of the left coronary artery 234 of a typically sized graft. FIG. 11 shows the dilated native aorta with a radius R1. Further, FIG. 12 shows the dilated native aorta with the dimensions of a typically sized graft according to existing grafts and existing methods of implantation superimposed to show the dimensional discrepancy between the radius or diameter of the graft (R2 and R3) and diameter of the dilated native aorta (R1) and, thus, the locations of the origins of left and right coronary arteries 232, 234.

Referring to FIGS. 8-10, the typical anatomy of a patient presenting with pathologic aneurysmal dilatation of the ascending aorta 230 and aortic root. The pathologic aneurysmal dilatation of the ascending aorta 230 is characterised by dilatation of the aortic root, dilatation of the ascending aorta 230 and effacement at the junction between ascending aorta and aortic root, also known as the sino-tubular junction (STJ). It will be apparent that pathologic dilation of the aorta 230 compresses adjacent anatomical structures forcing them progressively outwards and causing closely related anatomical structures, such as the origins or ostia of the right and left coronary arteries 232, 234, to adopt anatomical locations that are progressively further away from the central axis of the aorta 230.

FIG. 11 shows pathological dilatation of the aorta 230 of FIGS. 8-10 in cross section at the level where the coronary arteries 232, 234 arise from the aorta 230. The intersection of the dotted lines depicts the central axis of the aorta 230 at this level, for illustrative purposes. FIG. 11 also shows the left and right coronary arteries 232, 234 arising from the aorta 230. The angle subtending the origins of left and right coronary arteries 232, 234 is commonly less than 180 degrees. The distance between origin of the coronary arteries 232, 234 and the central axis of the graft 100 equates to the radius of the aorta 230, at this level (R1).

Referring now to FIG. 12, the radii of currently available aortic root replacement grafts, have a second radius (R2) representing the radius of the tubular portion of the graft 100 and a third radius (R3) of the bulged portion of the graft. It will be observed that there is a discrepancy, between the radii (R2 & R3) and the first radius (R1) of the pathologically dilated native aorta 230.

Referring now to FIGS. 14-19, the aortic root replacement graft 100 includes a bulge 130 in the graft 100 to accommodate the functions of the natural sinuses of Valsalva. Secondly, the graft 100 has tube grafts 160, 180 attached as side arms 160, 180 during manufacture of the aortic root replacement graft 100 substitute whose main purpose is to bridge the gaps 204, 206 between ARR graft 100 and the origins of the right and left coronary arteries 232, 234. As outlined in the methods below the side arms 160, 180 may be trimmed to an appropriate length by the surgeon.

The attachment of the aortic valve leaflets to the native aortic annulus is crown shaped. Typically, in the ARR procedure, sutures are placed along the edge of this crown and then passed through the aortic valve substitute and then through the base of the graft 100, otherwise, the valve is attached to the graft 100 as a valved graft composite and sutures may then be passed through the sewing cuff of the prosthetic valve alone. The effect of this is that the native aortic annulus and the sewing cuff of the valve prosthesis or lower border of the aortic root replacement graft are drawn together and into one plane as sutures are tied and said plane has a tendency to rise up towards the level of the tips of the crown.

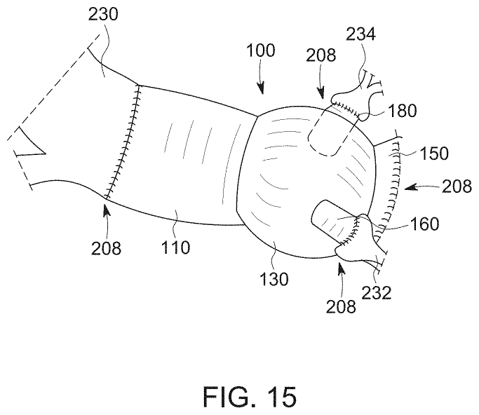

Referring now to FIGS. 14-15, the aortic root replacement graft 100 is implanted and includes suture lines 208 between the coronary arteries 232, 234 and the side tubes 160, 180, between the base 150 of the graft 100 and the aortic annulus where the aorta 230 exits the heart 220, and between the aortic root replacement graft substitute 100 and the distal portion of the aorta 230. The suture lines 208 need to be inspected carefully as bleeding or late aneurysm formation can occur at any of these various suture lines (anastomoses). As shown in FIG. 14, the graft 100 is implanted in the chest to adjacent anatomical structures following implantation. When the graft 100 is implanted the space between the new graft 100 and the surrounding anatomical structures remains. The space typically fills with fluid.

The graft 100 may be implanted into a patient by the following method. Anesthetise and prepare the patient for open heart surgery in the normal or known manner. Expose the heart by a median sternotomy or other suitable incision, such as partial upper sternotomy, minimally invasive right anterior thoracotomy or even less invasive transcervical incision and exposure via the neck. Optionally place pericardial stay sutures to improve exposure. Dissect the area between the aorta and the main pulmonary artery and its right branch as it passes behind the ascending aorta in order to provide mobilisation and better exposure around middle portion of the ascending aorta. Next, the surgeon may optionally pass a tape around the aorta to aid manipulation of the ascending aorta. The dissection may then proceed distally around the aorta at the junction between the ascending and aortic arch in preparation for the distal anastomosis of the aortic root replacement graft. Heparin is then administered in the normal manner and adequacy of heparinisation tested by measurement of the activated clotting time, ACT, or equivalent. After the heparin is administered, an arterial cannula is placed in the aortic arch or remotely in one of the peripheral arteries in the standard manner.

Next, a two-stage venous cannula is placed in the right atrial appendage or else bicaval cannulas are placed to collect the venous return. Alternatively, a long venous cannula may be passed from the groin via the femoral vein for venous drainage. Cardiopulmonary bypass is then established using standard techniques and according to standard parameters. The heart may then be optionally vented with a left ventricular vent or pulmonary artery vent or other venting technique. After venting, the aorta is cross clamped and cardioplegia administered in the normal manner using antegrade or retrograde myocardial protection technique or a combination of the above. The aorta is opened and the inside of the aorta, the aortic root and aortic valve are inspected. Cardioplegia may optionally be delivered directly into the coronary ostia at appropriate intervals, as necessary and topped up at intervals according to the discretion of the surgeon.

Then, the distal aorta is optionally prepared first by trimming. The distal aortic stump may optionally be further prepared for the distal anastomosis by attachment of a strip of Teflon felt, pericardium or other suitable material to its external surface. The distally prepared aorta may optionally be marked in quadrants with marker pen to aid subsequent anastomosis to the aortic root replacement graft. In addition, sizers may also optionally be used to measure the size or calibre of the distal aorta.

Attention is next turned to the aortic valve. The surgeon makes the decision whether to replace the aortic valve with a prosthetic valve, either mechanical or biological, or to preserve the native aortic valve leaflets and repair the aortic valve using an aortic valve sparing technique. If the valve is to be replaced, then the surgeon excises the native aortic valve cusps taking care to preserve the integrity of the native aortic annulus. The surgeon dissects around the coronary arteries leaving a cuff of native aorta to create the so called ‘coronary buttons’ that will be attached to the side arms of the aortic root replacement graft. In doing this dissection, the surgeon works from inside and outside of the aorta to define the boundaries of the buttons, typically with Metzenbaum scissors or similar.

If the surgeon chooses to perform aortic valve replacement, the native aortic valve leaflets are excised, the area washed thoroughly with cold saline to remove all debris and sutures are placed around the aortic annulus. The aortic annulus is sized using sizers and an appropriately sized valve prosthesis and aortic root replacement graft of the current invention selected. The aortic root replacement graft may be soaked in saline to improve later haemostasis depending upon the material from which the graft has been manufactured. Sutures are passed through sewing cuff of valve and then through the base of the aortic root replacement graft in sequence. Alternatively, the aortic valve prosthesis may first be attached to the graft with a running suture so that interrupted sutures may then be passed through the sewing cuff of the valve and graft more easily. In a further alternative, the graft may be attached to the valve prosthesis during manufacture so that the surgeon need only pass sutures through the sewing cuff of the valve portion of the valved conduit. The valve and aortic root replacement graft or composite are parachuted into position and individual sutures are tied securely.

If the surgeon decides to repair the valve, then the aortic root is dissected to the junction between left ventricle and the start of the aorta, the so called ventriculo-aortic junction. The native sinuses are trimmed so as to leave a 5-8 mm cuff of native aorta adjacent to the line of attachment of each valve leaflet. Then, sutures are placed under the native valve leaflets around the aorto-ventricular junction, annular sutures. An appropriate size of aortic root replacement graft 100 is selected. The aortic root replacement graft may optionally be soaked in saline, as outlined above. The previously placed annular sutures are passed through the base of the aortic root replacement graft and tied. The native valve commissures are then drawn upwards inside the aortic root replacement graft of the current invention and attached at an appropriate level and at appropriate radial positions relative to the long axis of the graft. Commissures are typically arranged at 120 degree radial positions for a trileaflet aortic valve repair and 180 degrees apart for a bicuspid aortic valve repair. The retained native portion of aorta adjacent to the line of attachment of each valve leaflet is attached to the inside of the aortic root replacement graft of the current invention. The valve is tested with saline. Plication sutures or other leaflet repair technique may be used to assure adequate valve repair.

Whether the valve has been repaired or the valve has been replaced, the side arms of the aortic root replacement graft are trimmed to an appropriate length. The heart may optionally be filled with blood to provide more insight as to how the graft and side arms will lie in the distended state when the circulation is filled with blood under normal pressures. The left coronary button is attached to the side arm of the aortic root replacement graft using, for example Prolene sutures, and the anastomosis inspected. The aortic root replacement graft may be distended with blood to check for haemostasis between the left coronary button and the side arm. However, a particular advantage of the graft 100 is that the coronary button suture lines remain accessible right to the end of the operative procedure so checks for haemostasis can reasonably be left until the end of the procedure for more efficient surgery. The right coronary button is attached to the respective side arm of the aortic root replacement graft and the suture line inspected and optionally tested for haemostasis in a similar manner as described for the left coronary button to left side arm anastomosis above.

Next, the aortic root replacement graft is trimmed to length for the distal anastomosis. The heart may optionally be distended with blood to determine the proper length of the aortic root replacement graft prior to performing the distal anastomosis. The reinforcing sleeve may then be placed over the aortic root replacement graft prior to performing the distal anastomosis or alternatively the distal anastomosis may be performed first and then the reinforcing sleeve cut in a longitudinal direction so that it may be passed around the distal anastomosis.

If the reinforcing sleeve is to be placed before construction of the distal anastomosis, it is placed over the tubular portion of the aortic root replacement graft prior to performing the anastomosis then its longitudinal length may be reduced by placing one or more encircling sutures so that the presence of the reinforcing sleeve does not obstruct execution of the distal anastomosis by the surgeon. If encircling sutures have been placed, then these may be cut at any convenient time after the distal anastomosis has been completed.

A venting chimney of the graft may be attached to a venting line from the cardiopulmonary bypass circuit and suction applied at, for example, 300 ml/min. De-airing drill according to the individual surgeon's preference is performed next. Once the surgeon judges that most of the air has been evacuated from the heart, the aortic cross clamp is released thereby restoring perfusion to the heart. Suction continues to be applied to the vent line attached to the venting chimney. The pressure in the circulation may be temporarily reduced so that the reinforcing sleeve may be drawn over the distal anastomosis.

Alternatively, if the reinforcing sleeve is to be placed after construction of the distal anastomosis, it is divided in a longitudinal direction and passed around the distal anastomosis. Whether placed before or after construction of the distal anastomosis, the proximal and distal portions of the reinforcing sleeve are tightened on either side of the distal anastomosis. The proximal and distal portions of the reinforcing sleeve may either be tightened temporarily with tourniquets or alternatively they may be tightened permanently by, for example, tying. If tourniquets were used then these may be temporarily released at intervals to allow for careful inspection of the distal anastomosis prior to permanent tightening by for example tying.

When the heart has recovered, the patient is weaned from cardiopulmonary bypass so that the heart takes over the circulation. Arterial and venous cannulas and vents are removed and sites oversewn in the normal manner. The venting chimney is tied at its base to close off the venting chimney from the circulation. The venting chimney may be oversewn beyond the tie for added security, at the discretion of the surgeon. Excess, redundant length of the venting chimney may be excised and discarded. Heparin is reversed with Protamine. The suture lines are inspected. Haemostasis is then secured. Finally, the chest is closed in routine manner according to incision type.

As may be recognized by those of ordinary skill in the art based on the teachings herein, numerous changes and modifications may be made to the above-described and other embodiments of the present disclosure without departing from the scope of the disclosure. The components of the grafts as disclosed in the specification, including the accompanying abstract and drawings, may be replaced by alternative component(s) or feature(s), such as those disclosed in another embodiment, which serve the same, equivalent or similar purpose as known by those skilled in the art to achieve the same, equivalent or similar results by such alternative component(s) or feature(s) to provide a similar function for the intended purpose. In addition, the grafts may include more or fewer components or features than the embodiments as described and illustrated herein. For example, the components and features of reinforcing sleeve 200, venting chimney 210, sizers, and valves may be used interchangeably and in alternative combinations as would be modified or altered by one of skill in the art in combination with the graft 100 including a tubular portion 110, a bulged portion 130, a cuff portion 150 and at least one arm 160, 180. Accordingly, this detailed description of the currently-preferred embodiments is to be taken in an illustrative, as opposed to limiting of the disclosure.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has”, and “having”), “include” (and any form of include, such as “includes” and “including”), and “contain” (and any form of contain, such as “contains” and “containing”) are open-ended linking verbs. As a result, a method or device that “comprises,” “has,” “includes,” or “contains” one or more steps or elements possesses those one or more steps or elements, but is not limited to possessing only those one or more steps or elements. Likewise, a step of a method or an element of a device that “comprises,” “has,” “includes,” or “contains” one or more features possesses those one or more features, but is not limited to possessing only those one or more features. Furthermore, a device or structure that is configured in a certain way is configured in at least that way, but may also be configured in ways that are not listed.

The disclosure has been described with reference to the preferred embodiments. It will be understood that the architectural and operational embodiments described herein are exemplary of a plurality of possible arrangements to provide the same general features, characteristics, and general system operation. Modifications and alterations will occur to others upon a reading and understanding of the preceding detailed description. It is intended that the disclosure be construed as including all such modifications and alterations.

What is claimed is:

1. An aortic root replacement graft, comprising:
a tubular portion;
a bulged portion with a first end and a second end, wherein the second end of the bulged portion is coupled to a first end of the tubular portion;
a cuff portion coupled to the first end of the bulged portion; and
at least one side arm coupled to and extending from the bulged portion.

2. The graft of claim 1, wherein the at least one side arm is a first side arm and a second side arm.

3. The graft of claim 2, wherein the first side arm and second side arm are disposed in a downward direction relative to a horizontal plane traversing the bulged portion of the graft.

4. The graft of claim 2, wherein the first side arm and the second side arm are flexible, and wherein the first side arm and the second side arm are bendable at least one of upwards, downwards, left and right in a cross-sectional plane of the first side arm and the second side arm.

5. The graft of claim 2, the first side arm and the second side arm further comprise:
at least one marker positioned between a first end and a second end of each of the first side arm and the second side arm.

6. The graft of claim 5, wherein the at least one marker is a plurality of markers dividing the circumference of each of the first side arm and the second side arm into quadrants.

7. The graft of claim 5, wherein the at least one marker is a plurality of markers dividing the circumference of each of the first side arm and the second side arm into thirds.

8. The graft of claim 1, further comprising:
a heart valve prosthesis coupled to an end of the graft positioned at or below the bulged portion.

9. The graft of claim 8, wherein the heart valve prosthesis is selected from at least one of a mechanical prosthesis and a biological prosthesis.

10. The graft of claim 2, further comprising:
a venting chimney coupled to and extending from an exterior surface of the graft.

11. The graft of claim 10, wherein the venting chimney is disposed perpendicular to a long axis of the graft.

12. The graft of claim 10, wherein the venting chimney is coupled to and extending from the tubular portion of the graft.

13. The graft of claim 10, wherein the venting chimney, the first arm, and the second arm are each radial offset around the tubular portion of the graft.

14. The graft of claim 1, further comprising:
a reinforcing sleeve positioned to engage a second end of the tubular portion.

15. The graft of claim 14, wherein the reinforcing sleeve is a straight tube with an internal surface having a first diameter and an exterior surface of the tubular portion having a second diameter, wherein the first diameter is larger than the second diameter.

16. The graft of claim 14, wherein the reinforcing sleeve includes a proximal portion with a first radius and a distal portion with a second radius, and wherein the first radius is different than the second radius.

17. The graft of claim 14, wherein the reinforcing sleeve comprises:
at least one encircling ligature.

18. The graft of claim 17, wherein the at least one encircling ligature is a first ligature and a second ligature, and wherein the first ligature is disposed on an end of the reinforcing sleeve with a larger radius.

19. The graft of claim 17, wherein the at least one encircling ligature is a first ligature and a second ligature, and wherein the second ligature is disposed on an end of the reinforcing sleeve with a smaller radius.

20. A method of implanting an aortic root replacement graft, comprising:
preparing a patient for open heart surgery;
obtaining the graft;
coupling a second end of the graft to a distal aorta;
coupling a first end of the graft to at least one of a proximal aorta and an aortic annulus;
coupling a first side arm to a first coronary artery;
coupling a second side arm to a second coronary artery; and
completing the open heart surgery and closing the patient.
